(19) United States
(12) Patent Application Publication
(10) Pub. No.: US 2015/0374537 A1
(43) Pub. Date: Dec. 31, 2015

(54) MRI-SAFE PATIENT THERMAL MANAGEMENT SYSTEM

(71) Applicant: IRADIMED CORPORATION

(72) Inventor: Roger Susi, Winter Park, FL (US)

(21) Appl. No.: 14/755,285

(22) Filed: Jun. 30, 2015

Related U.S. Application Data
(60) Provisional application No. 62/019,256, filed on Jun. 30, 2014.

(51) Int. Cl. A61F 7/00

(52) U.S. Cl. CPC A61F 7/0097; A61F 7/007; A61F 2007/0056; A61F 2007/0076; A61F 2007/0095

(57) ABSTRACT

Systems and methods for thermal management of a patient in an MRI environment are disclosed. The systems include a non-magnetic pump that provides fluid through fluid flow channels to a patient in the MM environment. In one embodiment, a control system adjusts the temperature or rate of flow of fluid provided to the patient in order to maintain or adjust the patient's temperature. In one embodiment, a display unit provides information on the temperature of the patient or the fluid and enables a user to adjust parameters of operation.

### FIELD

Embodiments generally relate to systems for pumping fluid (e.g., gas, such as ambient air, or liquid) to a patient thermal management blanket in a magnetic resonance image (MRI) environment of high magnetic fields with required low radiofrequency interference.

### BACKGROUND

It is desirable to maintain normal body temperature, especially for those receiving medical care. Warming and cooling blankets can be used to maintain appropriate patient temperature in a hospital. However, warming and cooling blankets and support equipment may contain elements that are dangerous, or will not operate effectively, in an MRI environment.

### SUMMARY

An MM-safe patient thermal management system may be used to control the temperature of a patient. In one embodiment, a fluid circulating blanket has a plurality of fluid (e.g., liquid or gas) flow channels. A thermal control unit monitors the temperature of the circulated fluid or additionally the patient, and controls the temperature of fluid that is delivered by tubing to the fluid flow channels. For example, the patient temperature may be obtained directly via an MM-safe temperature monitoring device (e.g., temperature sensor) within the thermal control unit or, alternatively, via interface to a separate MRI-safe temperature monitoring device. The thermal control unit delivers the temperature-controlled fluid using a non-magnetic pump. In one embodiment, the non-magnetic pump is operated by a non-magnetic ultrasonic actuator (e.g., motor). In another embodiment, the non-magnetic pump comprises a deflecting piezoelectric diaphragm. A heating and cooling assembly controls the temperature of the fluid to be delivered. In one embodiment, the heating and cooling assembly is a Peltier thermoelectric system.

In some embodiments, the patient thermal management system includes a display. The display may present the patient's temperature to a user, as well as, include one or more input devices for the user. For example, the display may include inputs enabling the user to adjust a set temperature for the patient or the fluid. Based on the inputs, the thermal control system may adjust its operating parameters.

The ultrasonic motor or piezoelectric diaphragm pump, constructed of non-magnetic materials is driven by a power source. These piezoelectric ultrasonic motors do not produce detrimental magnetic fields and are not affected by external magnetic fields. The ultrasonic motor may drive a peristaltic, diaphragm, or other suitable fluid pumping mechanism, while the piezoelectric diaphragm directly acts upon the fluid. The motors may be driven by an electronic signal with little RF harmonic noise in the spectral range of about 6 MHz to about 130 MHz in which MRI receivers are most sensitive.

The heating and cooling assembly may include a heat sink to help control the temperature of the fluid delivered by the system. In one embodiment, a fan is provided to provide air flow to the heat sink for improved efficiency. The fan may be operated by a second ultrasonic motor that is driven using similar parameters to the first ultrasonic motor to reduce RF interference.

For purposes of summarizing the disclosure, certain aspects, advantages, and novel features have been described herein. It is to be understood that not necessarily all such advantages may be achieved in accordance with any particular embodiment. Thus, the apparatus, methods, and systems described herein may be embodied or carried out in a manner that achieves or optimizes one advantage or group of advantages as taught herein without necessarily achieving other advantages as may be taught or suggested herein.

In an embodiment, a patient thermal management system comprises a fluid-circulating blanket having a plurality of fluid flow channels, a thermal control unit configured to control a temperature of fluid to be delivered to the plurality of fluid flow channels, and fluid tubing coupled between the thermal control unit and the fluid-circulating blanket. The thermal control unit may comprise a fluid reservoir, a non-magnetic pump configured to pump the fluid through the fluid tubing to the plurality of fluid flow channels, and a heating and cooling assembly coupled between the fluid reservoir and the non-magnetic pump.

In an embodiment, the patient thermal management system may comprise an interface comprising a display configured to display a temperature reading of the patient and one or more actuable inputs configured to enable an operator to change operating parameters of the system. The patient thermal management system may comprise a power supply configured to provide power to the non-magnetic pump. In an embodiment, the power supply may be configured to supply a substantially sinusoidal alternating current with minimal harmonic frequencies in the range of 6 MHz to 130 MHz.

In an embodiment, the heating and cooling assembly of a patient thermal management system may comprise a thermoelectric cooler. In some embodiments, a thermoelectric cooler may comprise a heat sink or heat shunt, a fan, and a non-magnetic ultrasonic motor coupled to the fan to control the operation of the fan. In some embodiments, the ultrasonic motor coupled to the fan may be powered by a power supply configured to provide approximately 10-24 peak to peak volts at approximately 1000 W.

In an embodiment, the non-magnetic pump of a patient thermal management system may comprise a piezoelectric diaphragm pump. In an embodiment, non-magnetic pump of a patient thermal management system may comprise a non-magnetic ultrasonic motor coupled to a non-magnetic pump to control operation of the non-magnetic pump.

### BRIEF DESCRIPTION OF THE DRAWINGS

FIG. 1 is schematic block diagram of an embodiment of a system for controlling the temperature of a thermal blanket.

FIG. 2 illustrates an embodiment of a control panel and display unit for a patient thermal management system.

FIG. 3 is a schematic drawing of an embodiment of a warming blanket as used in an MRI environment.

FIGS. 4A and 4B depict schematic diagrams of embodiments of a system for controlling the temperature of a thermal blanket.

FIG. 5 is a schematic diagram of an embodiment of control logic for a thermal control system.

### DETAILED DESCRIPTION

### Considerations for MRI Environment

The high magnetic field surrounding MM systems can negatively affect the operation of various devices, especially those devices that are constructed with magnetic materials. Those devices may also seriously jeopardize a patient's safety as a result of devices utilizing magnetic materials that can be attracted at high velocity into the magnetic field where a patient or attendant personnel are located.

Medical devices intended to be used within the MRI environment may require special consideration. RF stimulation of atomic nuclei within an associated magnetic field results in the emission of a small RF spin echo from the nucleus so stimulated. In the case of patient imaging, hydrogen nuclei bound with water are the usual targets for magnetic resonance at selected frequencies. Other molecules and compounds can also be selected for study, as in Nuclear Magnetic Spectroscopy, by choosing resonance specific magnetic field strengths and associated radio frequencies. For simplicity, the typical hydrogen atom-based image-acquisition process is referred to herein, but it should be recognized that the disclosure is equally useful in spectrographic studies at a plurality of field strengths and frequencies.

Certain devices may be needed in the MM scan room either to assist with care of the patient being imaged or for the use of attending staff. Of particular interest are those devices placed in the scan room during the time of image acquisition when the patient is present and the magnetic fields are up or active and RF reception of the relatively small nuclear echoes must be cleanly acquired. Electrically passive metallic items such as oxygen bottles or crash carts may present safety hazards to the patient due to their potential to be strongly attracted by the magnetic field of the scanner. Such items can be pulled into the imaging volume where the patient is located, creating potential for serious injury or death. Additionally, great effort is made during the manufacture and installation of the scanner/magnet to assure that the lines of flux within the imaging volume are highly homogenous to assure that acquired images have minimal spatial distortion. Thus, devices formed of magnetic material that are positioned within the magnetic field of the scanner can introduce distortions into this homogeneous field and the resultant images. The level of hazard and the degree of field/image distortion due to magnetic materials depends upon the composition and location with respect to the imaging volume.

The hazards due to flying objects can be controlled to some degree by the use of non-ferrous materials. Additionally, the gravitational weight of some devices or their rigid fixation in the scanning room may be sufficient to overcome the force of magnetic attraction on the ferrous mass of such devices toward the imaging volume. However, such devices with some ferrous mass, though inhibited from being pulled into the magnetic field, may nevertheless introduce inhomogeneity in the magnetic field. In accordance with several embodiments, distortions in the homogeneity of the magnetic field within the imaging volume must be kept at such a level as to be of minimal consequence to the operator reading the resultant image or data. The possibility of field distortion is proportionally increased as devices with metallic materials are positioned closer to the imaging volume, with the most critical position being near the center of the imaging volume, essentially where the patient is positioned.

Additionally, because of the extremely low levels of RF signals produced by the target image nuclei, great care may be taken to assure that devices with active electronic circuits do not emit spurious RF signals as forms of electronic noise. Such noise can so degrade the signal-to-noise ratio of signals received by the sensor coils and receivers that image resolution is reduced or rendered completely unreadable. Active circuits may be carefully shielded to assure that their RF emissions are extremely low at the specific frequencies of the imaging process. Conversely, it is possible through careful design, to place electrical circuits for the operation of medical devices, or the like, within the MRI environment, but such circuits may be designed to avoid the discreet Larmor frequencies unique to the particular magnetic field strength of a given scanner. The intense magnetic fields produced by the scanner can cause detrimental effects on the performance of common AC, DC and stepper motors in devices needed within the scanning room, to the point of making their control difficult or causing their complete failure. The gradient or time-varying magnetic fields can induce changing (AC) currents in motors and associated circuitry which may also cause false motor operation. In some embodiments, shielding or other elements may comprise non-ferrous, electrically conductive materials such as brass, copper, alloys of stainless steel or other metals, conductively-coated plastic, and/or the like. Furthermore, RF filters may be utilized at locations where electrical signals pass from shielded areas to areas where internally-produced electromagnetic interference signals can enter the MRI scan room and cause distortion.

Presently available systems and devices for patient thermal management commonly utilize resistive heaters and refrigerant/compressor systems for cooling. Cooling with compressor systems in an MRI environment may introduce hazards if these systems and devices are composed of magnetic-type steels or other magnetic materials. In addition, the use of AC or DC motors may also create magnetic hazards and the motors may not function properly due to the effects of magnetic fields upon such motor types.

### Thermal Control System:

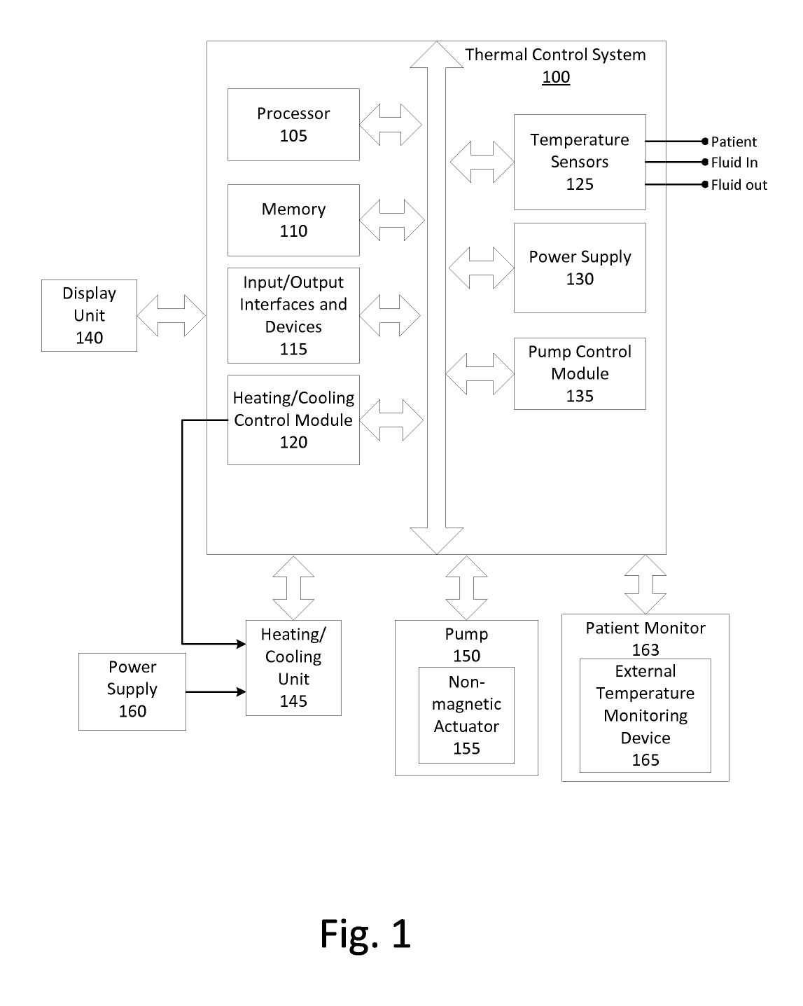

FIG. 1 shows a schematic block diagram of an embodiment of a thermal control system 100 that may be used to control the temperature of a blanket, covering, or body wrap used to warm and/or cool a patient. In some embodiments, the thermal control system 100 may include a display or may be connected to a separate display unit 140. The thermal control system 100 may also include a processor 105, memory 110, input/output interfaces and devices 115, a heating/cooling control module 120, one or more patient temperature sensors 125, a power supply 130, and a pump control module 135. The various components or modules of the thermal control system 100 may communicate with each other via a bus or other communication line or may communicate wirelessly.

The thermal control system 100 may be implemented using a single computing device or multiple computing devices. The processor 105 may be any hardware computing device, such as a central processing unit or microcontroller. In some embodiments, the processor 105 may analyze data from the temperature sensors 125 or signals from a pump 150 to determine operational parameters for the pump 150, a non-magnetic actuator 155 (e.g., motor), and/or a heating/cooling unit 145. The processor 105 may also communicate with the display unit 140 to output data or information to a user and accept user commands or other input via the input/output interfaces and devices 115 (e.g., an input keypad, a remote control, or other input devices).

In accordance with several embodiments, the processor 105 is configured to communicate with the memory 110. The memory 110 may contain computer program instructions (organized into modules) that the processor 105 executes in order to implement one or more embodiments of the present disclosure. The memory 110 may include RAM, ROM, other persistent or non-transitory computer-readable media, or some combination of memory elements. The memory 110 may store an operating system that provides computer program instructions for use by the processor 105 in the general administration and operation of the processor 105. The memory 110 may further include other information for implementing aspects of the present disclosure. One or more modules of thermal control system 100, such as the heating/cooling control module 120 or the pump control module 135, may be implemented by the processor 105 performing instructions stored as modules in the memory 110.

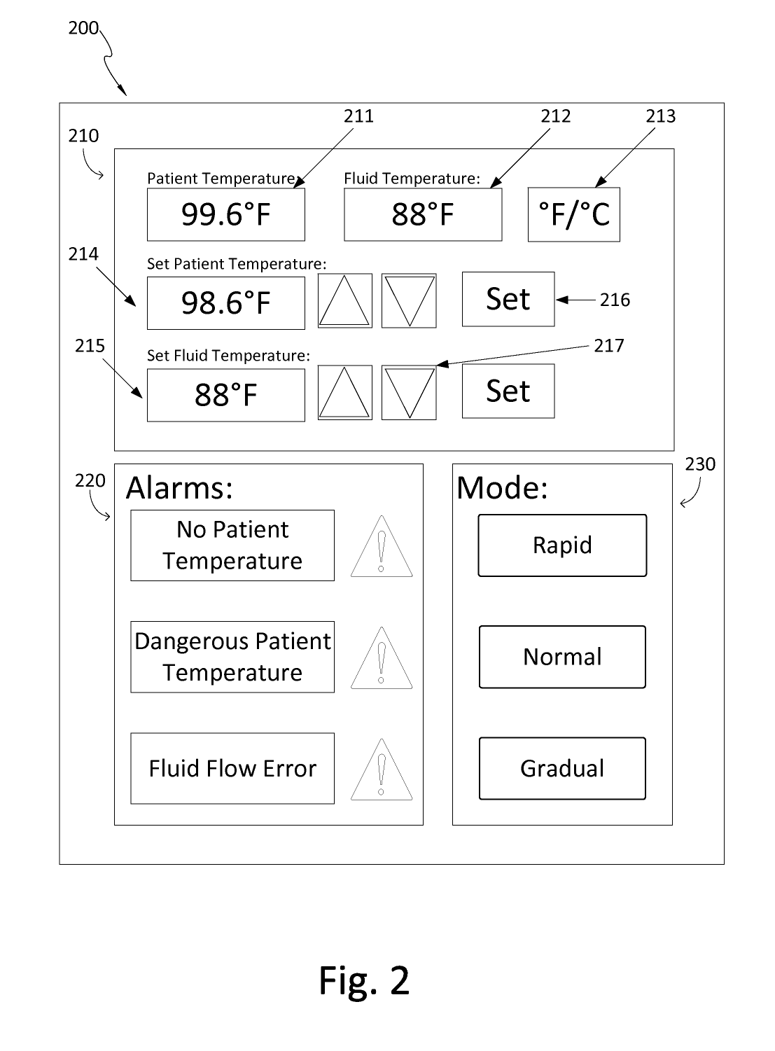

The input/output interfaces and devices 115 may include one or more input ports, including, but not limited to, keyboard or keypad ports, Bluetooth or other wireless links, optical ports, USB ports, and/or the like. The input/output interfaces and devices 115 may accept input from or one or more input devices, including, but not limited to, keyboards, mice, trackballs, trackpads, joysticks, input tablets, track points, touch screens, remote controls, velocity sensors, voltage or current sensors, motion detectors, or any other input device capable of obtaining a temperature, rate, or magnitude limit value from a user. In some embodiments, the input devices may include devices or connectors configured to receive power from a power supply 160 of a thermal control system and provide power to the heating/cooling unit 145, the pump 150, or other elements of the system. The input/output interfaces and devices 115 may accept input from a user indicating a desired temperature of the patient, warming/cooling blanket temperature, and rate of warming/cooling, and may display information such as the blanket's temperature, temperature of fluid pumped into the blanket, and the temperature of the patient. An example display unit with control buttons or other input mechanisms (e.g., touch screens, switches) to accept user input is discussed below in reference to FIG. 2.

The input/output device interfaces and devices 115 may also provide output via one or more output devices, including, but not limited to, one or more speakers optical ports, wireless interface, serial or USB ports. In some embodiments, the input/output device interfaces and devices 115 may include an interface to an MRI system or monitoring device.

The temperature sensors 125, 340, 470, and 480 may include sensors attached to various points in a fluid delivery system. For example, sensors may determine (e.g., read, monitor, etc.) the temperature of the patient, the warming blanket, fluid exiting the blanket, the fluid leaving the heating/cooling unit 145, or the temperature at other points in the system. The temperature sensors may be of any type capable of producing accurate readings, such as a resistance temperature detector, a thermistor, or a thermocouple. In one embodiment, a method of measuring the patient temperature within the MM bore is performed using a fiber optic temperature device 340 to avoid risk, or reduce the likelihood, of high RF heating associated with conductive devices in the bore. For example, the fiber optic temperature device may be designed such that it will not resonate at the RF frequencies produced by an imaging device. This may reduce the likelihood of the temperature sensor from picking up the RF stimulus energy, heating up, and potentially burning a patient. The temperature sensors 125 may supply temperature readings to the processor 105, the heating/cooling control module 120, and/or the pump control module 135 to be used to determine operating parameters. The temperature readings may also be provided to the display unit 140 for display to a user.

In some embodiments, the thermal control system 100 may receive temperature readings from an external temperature monitoring device 165 (for example, via the input/output interfaces 115). In some embodiments, such a monitoring device may be integrated into an MRI patient monitor 163 or another system operating in the MRI environment. For example, an MM patient monitoring system may include various monitoring devices for heart rate, pulse, respiratory rate, oxygen levels, blood pressure, blood oxygenation levels, and/or the like. The temperature readings from the patient monitoring system may then be provided to the temperature sensor 125. For example, the readings may be sent by wireless, fiber optic, or other means to the thermal control system 100. The readings from the external temperature monitoring device 165 may be used in addition to or as an alternative to readings from temperature sensors 125 of the thermal control system 100. In some embodiments, one or more temperature sensors 125, 340, 370, 380 or the external temperature monitoring device 165 comprise fiber optic temperature sensors or other MRI-safe temperature sensors configured to safely operate in an MRI environment without interfering with the imaging systems.

The pump control module 135 and the heating/cooling control module 120 may be implemented in hardware or software in the thermal control system 100. For example, the modules may be implemented by computer instructions stored in memory 110 and executed by the processor 105. The pump control module 135 may operate the pump 150 by controlling operation of a non-magnetic actuator 155 (e.g., an ultrasonic motor or a piezoelectric diaphragm), or the pump control module 135 may directly operate a piezoelectric diaphragm pump. For example, the pump control module 135 may determine the speed (if the non-magnetic actuator 155 is a motor) or a diaphragm deflection rate (if the non-magnetic actuator 155 is a piezoelectric diaphragm), to meet the current flow requirements of the system. The heating/cooling control module 120 may operate the heating/cooling unit 145 to set the parameters so that fluid leaving the heating/cooling unit 145 is set and/or maintained at a desired temperature to be passed to the blanket.

The heating/cooling unit 145 may warm or cool fluid to a desired temperature for the user. The heating/cooling unit 145 may include various heating elements to warm fluid, and heat sinks, heat shunts, or other systems to cool fluid. In some embodiments, the heating/cooling unit 145 is capable of both heating and cooling fluid depending on the system's needs. In some embodiments, the heating/cooling unit 145 may only perform one of heating or cooling.

The pump 150 is configured to provide fluid that is warmed or cooled by the heating/cooling unit 145 to a thermal blanket. The pump 150 may include the non-magnetic actuator 155 that is used to move fluid (e.g., gas or liquid) through the system. In some embodiments the non-magnetic actuator 155 may be an ultrasonic motor. In other embodiments, the non-magnetic motor 155 may be another type of motor that is capable of operating in an MM environment and does not present a danger to the patient or interfere with MRI readings. In some embodiments, the actuator 155 comprises a non-magnetic piezoelectric diaphragm pump member configured to pump fluid to the heating/cooling unit 145 based on outputs from the thermal control system 100.

The housing of the thermal control system 100, and other elements of the system may include shielding or filtering materials or devices to prevent spurious emissions of radiofrequency energy that could potentially distort or degrade images obtained by MM equipment.

In some embodiments, some or all of the electronic circuits present in the thermal control unit 100 or related systems discussed above may advantageously be shielded to reduce the potential impact on the sensors associated with the imaging device. For example, control circuits, power circuits, and other active electronics may be shielded by conductive structures disposed around the circuits. This may inhibit any potential RF signals output by the circuits. Furthermore, the shielding may prevent the fields generated by the imaging device from interfering with proper operation of the electronic circuits. In some embodiments, the electronic circuits may be designed with filters which prevent the buildup of RF signals on the electronic circuits to further protect the control systems from interference from the imaging device. Furthermore, in some embodiments, the circuits may be designed to avoid the specific Larmor frequencies or other frequencies of radiation that are used by the imaging device, thus further reducing the potential interference with the imaging sensors.

### Display Unit

FIG. 2 is an example embodiment of a display unit with control buttons for accepting user input. The display and control unit 200 shown in FIG. 2 may be implemented with LEDs, LCD screen(s), push buttons, and/or other physical inputs and outputs for a user to interact with. In some embodiments, the display and control unit 200 shown in FIG. 2 may be implemented as a user interface on a remote control device, adjunct monitor system, or computer system. For example, a user may view the user interface on a tablet, smartphone, or computer screen, and may interact with various buttons and actionable elements through a mouse, voice command, and/or touch screen depending on the embodiment. The display and control unit 200 may be controlled by the thermal control system 100. In some embodiments, the display unit may be formed of a non-magnetic, RF-shielding material, such as a conductively-coated plastic or aluminum, or the like, around the control systems or other electronic circuitry. This may reduce the level of RF transmissions from the electronic circuitry while also providing a safe display system which will not interfere with the operation of the imaging device.

In some embodiments, the display and control unit 200 is integrated into a front panel of the thermal control system 100. In other embodiments, the display and control unit 200 may be a separate and distinct component accessible remotely. For example, the display and control unit 200 may be operated from outside of the MRI environment and may communicate with the thermal control system 100 through wired or wireless communications (e.g., through RF signals).

In the example embodiment of FIG. 2, display and control unit 200 includes three main sections: a thermal control section 210, an alarm section 220, and a mode selection section 230. In some embodiments, the display and control unit 200 may include fewer or additional sections and fewer or additional features. The sections may be included on one display, as shown, or may be displayed on multiple display units. For example, in some embodiments, there may be a display and control unit 200 on a front panel of the thermal control system 100, and there may be a second display and control unit 200 accessible at a remote location. In some embodiments, only a portion of the features of a display and control unit 200 may be accessible from certain locations.

The thermal control section 210 may display a reading of the patient's temperature 211 and the fluid temperature 212. In some embodiments, the system may only include one temperature reading, such as the patient's temperature. In other embodiments, the system may include additional temperature readings, such as readings of the temperature at one or more locations of the thermal management blanket. The Fahrenheit/Celsius button 213 enables a user to change between viewing temperatures in Fahrenheit or Celsius. In some embodiments, each temperature displayed may have a Fahrenheit/Celsius button 213 associated with it. In other embodiments, as shown in FIG. 2, one Fahrenheit/Celsius button 213 controls the display of all temperature readings.

The set patient temperature display 214 and set fluid temperature 215 fields enable a user to control the patient's temperature. For example, in FIG. 2, the patient's temperature is set to 98.6° F., and the fluid temperature is set to 88° F. In some embodiments, the user may only be able to set one temperature, for example, the patient's temperature. In some embodiments, the user may be able to adjust the patient's temperature or the fluid temperature, but both cannot be set at the same time. Temperature adjustment buttons 217 enable the user to adjust the set temperatures. Temperature set button 216 enables the user or operator to instruct the thermal control system 100 to control the temperature according to the set temperature.

The alarm section 220 displays alarms (e.g., warnings, cautions, or other messages or information) to the user. For example, as shown in FIG. 2, the display and control unit 200 may display alarms if there is no patient temperature reading, a dangerous temperature reading (e.g., too high or too low), or a fluid flow error. In some embodiments, there may be fewer or additional alarms. The alarms may include lighting one or more alarm indicators (e.g., LEDs). In some embodiments, the thermal control system 100 includes an audio alarm instead of, or along with, the visual alarms. In some implementations, the audio alarms may include generating an audible alarm sound and/or sending an alarm signal to a remote control/display. The mode selection section 230 enables the user to set a mode for warming or cooling a patient. For example, in FIG. 2 the user is able to select rapid, normal, or gradual warming or cooling of the patient, thus controlling thermal rate of change.

### Warming/Cooling System

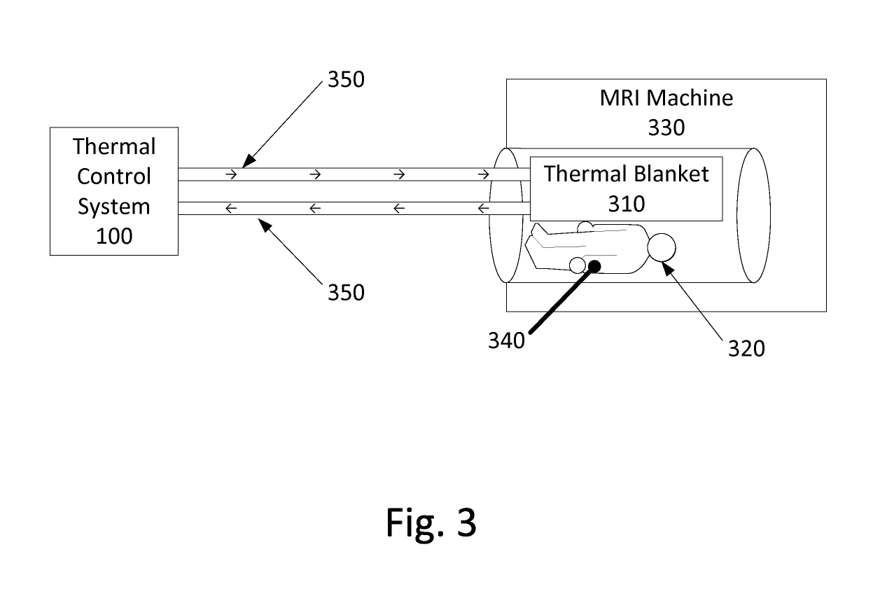

FIG. 3 illustrates one embodiment of a warming/cooling system. In FIG. 3, the thermal control system 100 provides fluid to the warming/cooling blanket 310. The fluid may be a liquid or a gas depending on the embodiment. As illustrated in FIG. 3, the patient 320 may use the thermal blanket 310 while in an MRI machine 330. The thermal blanket 310 preferably does not have magnetic aspects or RF conductive properties that would be dangerous for the patient or interfere with the MRI machine's imaging. The thermal control system 100 may be the system described in reference to FIG. 1, or may be another system capable of providing fluid (e.g., liquid or gas) to the thermal blanket 310 without interfering with the operation of, or images obtained by, the MRI machine 330 and/or without endangering the patient (e.g., without risking burning of the patient). The fluid may be provided to the thermal blanket 310 by one or more sets of fluid tubing 350 that provide fluid through the tubing forced by the pump. In some embodiments, the fluid may circulate in a closed circuit to and from the thermal blanket 310. In some other embodiments, the fluid may not return from the thermal blanket 310.

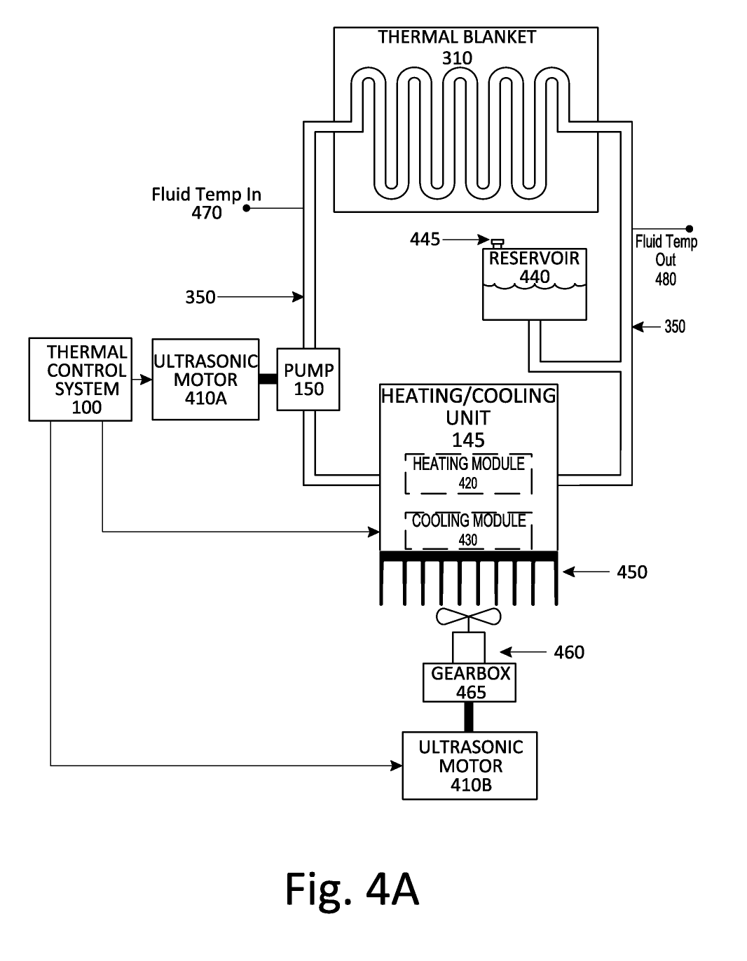

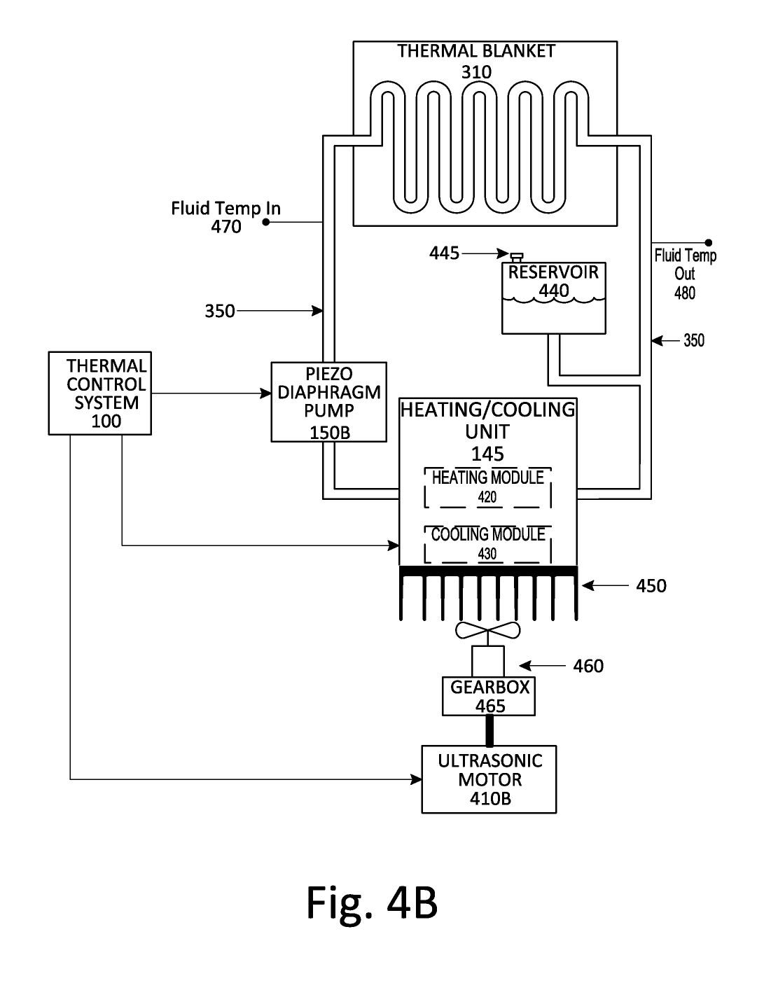

FIG. 4A is a schematic diagram of one embodiment of a warming and cooling system. The thermal control system 100 interacts with an ultrasonic motor 410A to operate the pump 150 as described with reference to FIG. 1 above. For example, the ultrasonic motor 410A may be driven with the pump control module 135. In some embodiments, in order to efficiently warm or cool a patient, the pump 150 may pump approximately 1.6 L/min. In other embodiments, the pump 150 may pump volume at a higher or lower rate, for example in the range of 0.5 L/min to 1.5 L/min, or in some embodiments greater than 2 L/min. The pump 150 may provide the fluid to the thermal blanket 310 which comprises fluid flow channels through fluid tubing 350. In some embodiments, the thermal blanket 310 returns fluid in a closed circuit through additional fluid tubing 350. Providing a closed circuit may increase the efficiency of heating or cooling the fluid as it passes through the heating/cooling unit 145.

The ultrasonic motor 410A may be driven by an electronic signal with little RF harmonic noise in the spectral range of about 8 MHz to about 130 MHz in which MRI receivers are most sensitive. In some embodiments, the drive power for the ultrasonic motor is generated via circuitry which produces multiphasic drive signals of at least sine and cosine waveforms at related ultrasonic frequencies. In other embodiments, single phase drive signals are used. In some embodiments, the drive signals are produced as a sinusoidal wave to reduce high frequency harmonic components that may disturb or hinder RF responsiveness.

One possible scheme for producing these multiphasic signals uses coreless or “Air Core” transformers constructed with inherent leakage inductance that interacts with the complex impedance of the ultrasonic motor to convert lower voltage square wave signals at the primary winding into sinusoidal high voltage signals at the secondary windings suitable for powering the ultrasonic motor and producing little harmonic RF interference. Alternatively, D.C. voltages of opposite polarities can be alternately switched to supply alternating voltages. The switched signals can be filtered into sinusoidal signals and applied to the inputs of high voltage linear amplifiers that are set for such gain as needed to produce resultant outputs of sufficient voltage and sinusoidal shape to drive the ultrasonic motor.

In accordance with one embodiment of a pump 150, very little or no magnetic material is used in any of the components of the pump including the ultrasonic motor 410A and associated components. Additionally, none of such components is adversely affected during operation by a strong magnetic field. Any RF energy that may be generated by electronic signals within the ultrasonic motor 410A, the thermal control system 100, or associated components may be specifically shielded by conductive structures disposed around such components to inhibit radiation of RFI. Additionally, radio-frequency interference filters are disposed about all through-shield conductors to inhibit radiation of RFI through such portals.

The pump 150 may pump fluid received from the heating/cooling unit 145. In some embodiments the heating/cooling unit 145 comprises a Peltier thermoelectric module. Peltier heating and cooling elements operate using the thermoelectric effect and can be designed with no magnetic materials and may operate within large magnetic fields. In the Peltier element, when current is passed through a semiconductor, heat is transferred from one side of the semiconductor to the other. The temperature difference from the warm side of a semiconductor to the other may be as much as 70° F. in the Peltier element. A Peltier module may be capable of removing approximately 200 W or more of heat from a system. In some embodiments the heating/cooling unit 145 comprising a Peltier module may be able to remove in the range of 40 W-360 W (e.g., 40 W-100 W, 65 W-120 W, 90 W-180 W, 100 W-300 W, 150 W-250 W, 200 W-300 W, 250 W-360 W, or overlapping ranges thereof) of heat from a system depending on the conditions and the requirements of the system. A large power supply 160 capable of supplying 500 W to over 1000 W may be used to supply such large capacity Peltier devices. The large power supply is designed with little or no magnetic materials to minimize, or reduce the likelihood of, magnetic attraction issues and operate properly and efficiently near the high magnetic fields produced in an MRI environment. The power supply may be connected to an alternating current source. In addition, the power supply may include a battery. In one embodiment, the battery comprises a non-magnetic lithium polymer battery or other battery made of a non-magnetic material. In order to effectively warm or cool a patient, the heating/cooling unit 145 may use fluid approximately 10-15° F. warmer or cooler than the patient's ambient temperature

In order for the Peltier module to operate efficiently, the heating/cooling unit 145 may have a heat sink 450 attached to the module. Additional heat sinking can be provided by careful design of the overall housing such that heat can be safely shed (during cooling mode) or gathered (during heating mode), directly from the unit housing, with or without forced air movement, and in such a way as to not expose users to excessively warm or cold surfaces. In some embodiments, the heat sink may be cooled by a fan 460 that is operated by an ultrasonic motor 410B. The ultrasonic motor 410B may be controlled by the thermal control system 100. The drive circuits of the ultrasonic motor 410B may be similar to those used in the operation of the ultrasonic motor 410A, as discussed above. In order to operate at speeds to effectively cool the heat sink 450, the ultrasonic motor 410B may be driven with a power supply capable of supplying AC power in the range of 10-24V and at powers up to 1000 W. In some embodiments the system includes a gearbox 465 which is used to drive the fan blades from a relatively slow ultrasonic motor 410B. In some embodiments, a gearbox or other elements may be integrated into the fan 460 in order to operate at the appropriate speeds for cooling the heat sink 450 while driven by the ultrasonic motor 410B.

In some embodiments using a Peltier module for the heating/cooling unit 145, the temperature may be controlled by thermal control system 100 by changing the polarity of the current through the semiconductor. For example, the thermal control system 100 may determine that it is necessary to cool a patient to bring that patient to the set temperature. The thermal control system 100 may cool the patient by providing a positive current through the Peltier module to cool the fluid to be pumped to the thermal blanket 310. At a later time, the thermal control system 100 may determine that to bring the patient back to the set temperature that it is necessary to warm the patient by pumping warm fluid through the thermal blanket 310. The thermal control system may accomplish the warming by switching the polarity of the current through the Peltier module so that the fluid will be heated before pumping to the thermal blanket 310.

In some embodiments, as depicted in FIG. 4B, a warming and cooling system may utilize a piezoelectric diaphragm pump 150B instead of a non-magnetic ultrasonic motor attached to a pump. The remaining components illustrated in the schematic diagram of one embodiment of a warming and cooling system shown in FIG. 4B may have similar attributes and features as described in reference to FIG. 4A above. Using a piezoelectric diaphragm pump may increase the lifespan of the thermal management system compared to an ultrasonic motor attached to a pump. In addition to the piezoelectric diaphragm pump and ultrasonic motor embodiments, the system may be configured to use other non-magnetic pumps and motors that are safe in an MRI environment and will not interfere with the imaging systems.

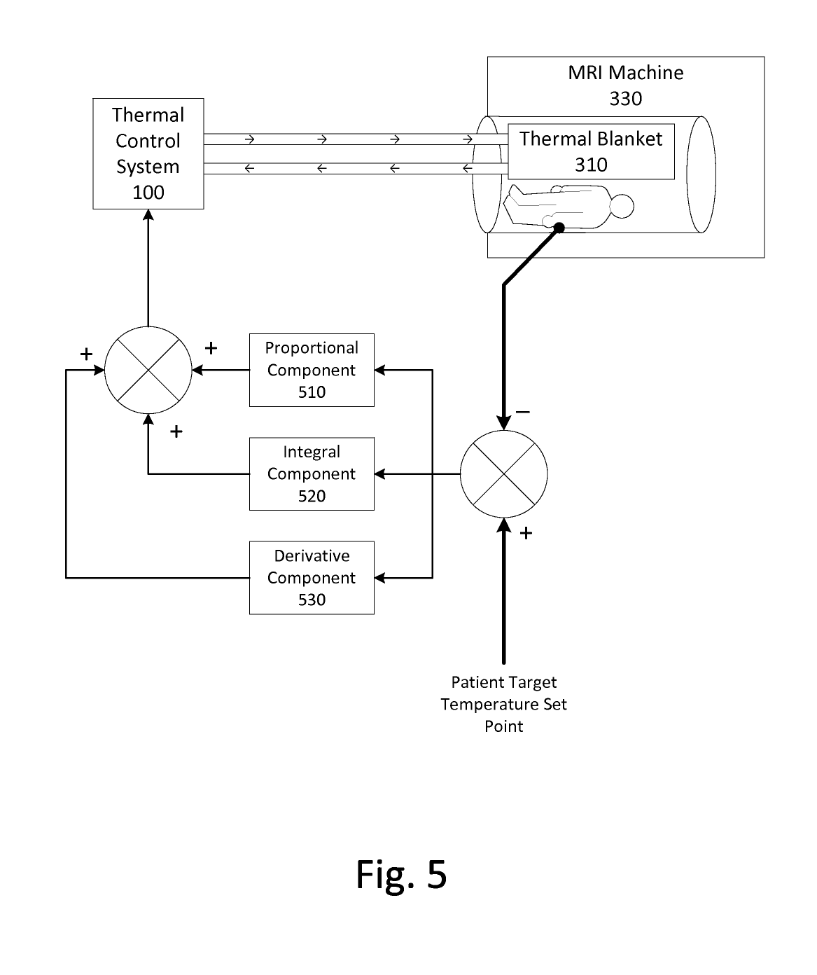

To maintain accurate temperature control, the thermal control system 100 may also include one or more control methodologies for loop control of the heating/cooling process as shown in FIG. 5. In a basic control implementation, the thermal control system 100 may comprise a feedback loop with a proportional component 510 that applies a constant proportional term to an error reading representing a temperature difference between a measured temperature and a set temperature value. In some embodiments, the set temperature value is set directly by a user. For example using the display and control unit 200 discussed in reference to FIG. 2 above. In some embodiments, the set temperature value is a desired temperature for the patient or a desired temperature of the fluid. In some embodiments, the measured temperature is the patient's temperature, the temperature of fluid leaving the heater, the temperature leaving the thermal blanket, or another temperature reading. In some embodiments, the thermal control system 100 may also include an integral component 520 as an additional term of a control feedback loop. Adding an integral component to the feedback loop may help speed up and stabilize the device's capacity to reach and hold the user given temperature set point. In some embodiments, the thermal control system 100 may also include a derivative component 530 as a term of the control feedback loop, which may further improve the performance of the control system. In various embodiments, the thermal control system 100 may include a combination of one or more of the discussed components. For example, the thermal control system 100 may include a feedback loop that has a proportional component and integral component, but no derivative component. The components of the thermal control system 100 may be implemented in hardware such as operational amplifiers or other hardware components. The control methods used thermal control system 100 may also be implemented as one or more software modules.

In other embodiments, the heating/cooling unit 145 may include a separate heating module 320 and a cooling module 330. The heating module 320 may contain one or more heating elements such as resistors to act as immersion heaters. The cooling module 330 may use heat sinks and a heat exchange to cool the fluid, using compression pumps, or may use other cooling techniques.

Fluid is pumped through the thermal blanket 310 to provide heating or cooling to the patient. The fluid is dispersed through channels in the blanket to warm or cool a sufficient surface area of the blanket. Various embodiments of the thermal blanket 310 may have channels in a variety of configurations for fluid to flow through. The fluid may be pumped through the thermal blanket 310 to return to the heating/cooling unit 145. The system may include a reservoir 440 which provides a consistent supply of fluid to the heating/cooling unit 145 and the pump 150 so that fluid may be continuously supplied to the thermal blanket 310. The reservoir 440 may include an opening 445 for receiving and/or replenishing circulating fluid. For example, the opening 445 may be connected to a fluid supply line or may provide an entry point for providing or replenishing fluid. In some embodiments, the opening 445 may open to the ambient air of the room, enabling the system to add additional fluid in the form of air to the system as needed for continued operation. The thermal blanket 310 may be made of any fabric suitable for use in a medical facility while containing the cooling/heating fluid. In some embodiments, the thermal blanket 310 may be disposable so that a new blanket can be attached to the system for each patient. The fluid channels may be made of a polymer or other material that will not collapse to stop the flow of fluid, but that bend to conform to the shape of a patient.

### Other Embodiments:

In some embodiments the heating/cooling fluid is water. However, in other embodiments, other fluids (e.g., liquids or gasses) may be used which provide advantageous characteristics. For example, air from the room may be used to warm or cool the thermal blanket, providing a readily available source of fluid for the device. In some embodiments, other non-magnetic systems may be used to provide heating, cooling, or pumping. For example, instead of an ultrasonic motor to drive the pump 150, the system may use another type of non-magnetic pump. In addition for use with a thermal blanket, the systems disclosed may be used to pump warmed or cooled fluids for other purposes. For example, a patient seeking an MRI may benefit from a cold pack to treat inflammation. Instead of pumping fluid to a thermal blanket, the disclosed system may be used to pump very cold water to a liquid bladder in a cuff that can be placed over the affected region.

In some embodiments, the patient thermal management system 100 may be used in non-MRI environments. For example, the ultrasonic motors and Peltier heating/cooling systems used in some embodiments of the system may be beneficial to users desiring a low noise system of providing thermal control to a patient. The thermal management system may be a portable system such that it can be transported with the patient for use continuous treatment before, during, and after the patient is in an MRI environment.

The foregoing disclosure has oftentimes partitioned devices and systems into multiple modules (or components) for ease of explanation. It is to be understood, however, that one or more modules may operate as a single unit. Conversely, a single module may comprise one or more subcomponents that are distributed throughout one or more locations. Furthermore, the communication between the modules may occur in a variety of ways, such as hardware implementations (for example, over a network, serial interface, parallel interface, internal bus, or the like), software implementations (for example, database passing variables), or a combination of hardware and software. It will be appreciated that modules may be sub-components of other modules, and multiple modules may share common components. For example, multiple modules may be implemented with firmware or software and share common hardware components, such as processors and memory.

A module can be, for example, logic embodied in hardware, software, firmware, or any combination of hardware, software, and/or firmware. Software instructions may be embedded in firmware, such as a ROM, EPROM or Flash memory. Software instructions may be written in a programming language, such as, for example, assembly, Java, Lua, Objective-C, C or C++, and may be compiled and linked into an executable program, installed in a dynamic link library, or may be written in an interpreted programming language such as, for example, BASIC, Perl, or Python. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware. Generally, the modules described herein refer to logical modules that may be combined with other modules or divided into sub-modules despite their physical organization or storage.

Each of the processes, components, and algorithms described above can be embodied in, and fully automated by, code modules executed by one or more computers or computer processors. The code modules can be stored on any type of computer-readable medium or computer storage device. The processes and algorithms can also be implemented partially or wholly in application-specific circuitry. The results of the disclosed processes and process steps can be stored, persistently or otherwise, in any type of computer storage. In one embodiment, the code modules can advantageously be configured to execute on one or more processors. In addition, the code modules can comprise, but are not limited to, any of the following: software or hardware components such as software object-oriented software components, class components and task components, processes methods, functions, attributes, procedures, subroutines, segments of program code, drivers, firmware, microcode, circuitry, data, databases, data structures, tables, arrays, variables, or the like.

Conditional language, for example, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

While the invention has been discussed in the context of certain embodiments and examples, it should be appreciated that the present invention extends beyond the specifically disclosed embodiments to other alternative embodiments and/or uses of the inventions and obvious modifications and equivalents thereof. Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Additionally, the skilled artisan will recognize that any of the above-described methods can be carried out using any appropriate apparatus. Further, the disclosure herein of any particular feature, aspect, method, property, characteristic, quality, attribute, element, or the like in connection with various embodiments can be used in all other embodiments set forth herein. Additionally, processing steps may be added, removed, or reordered. A wide variety of designs and approaches are possible.

For purposes of this disclosure, certain aspects, advantages, and novel features of the embodiments are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

What is claimed is:

1. An MRI-safe patient thermal management system comprising:
a fluid-circulating blanket having a plurality of fluid flow channels;
a thermal control unit configured to control a temperature of fluid to be delivered to the plurality of fluid flow channels; and
fluid tubing coupled between the thermal control unit and the fluid-circulating blanket,
wherein the thermal control unit comprises:
a fluid reservoir;
a non-magnetic pump configured to pump the fluid through the fluid tubing to the plurality of fluid flow channels of the fluid-circulating blanket; and
a heating and cooling assembly coupled between the fluid reservoir and the non-magnetic pump, the heating and cooling assembly configured to heat or cool the fluid to a specified temperature.

2. The MRI-safe patient thermal management system of claim 1, further comprising an interface coupled to the thermal control unit comprising:
a display configured to display a temperature reading of the patient;
one or more actuable inputs configured to enable an operator to change operating parameters of the system.

3. The MRI-safe patient thermal management system of claim 1, further comprising a power supply configured to provide power to the non-magnetic pump.

4. The MM-safe patient thermal management system of claim 3, wherein the power supply is configured to supply a substantially sinusoidal alternating current with minimal harmonic frequencies in the range of about 6 MHz to about 130 MHz.

5. The MRI-safe patient thermal management system of claim 1, wherein the heating and cooling assembly comprises a thermoelectric cooler.

6. The MRI-safe patient thermal management system of claim 5, further comprising:
a heat sink coupled to the heating and cooling assembly;
a fan configured to provide air flow to the heat sink; and
a non-magnetic ultrasonic motor coupled to the fan, the non-magnetic ultrasonic motor configured to control operation of the fan.

7. The MM-safe patient thermal management system of claim 6 further comprising a power supply configured to supply power to the non-magnetic ultrasonic motor, wherein the power supply is configured to supply 10-24 peak to peak volts at 1000 W.

8. The MM-safe patient thermal management system of claim 1, wherein the non-magnetic pump comprises a piezoelectric diaphragm pump.

9. The MRI-safe patient thermal management system of claim 1, further comprising a non-magnetic ultrasonic motor coupled to the non-magnetic pump, the ultrasonic motor configured to control operation of the non-magnetic pump.

10. An MRI-safe patient thermal management system comprising:
a fluid-circulating blanket having a plurality of fluid flow channels;
a thermal control unit configured to control a temperature of fluid to be delivered to the plurality of fluid flow channels; and
fluid tubing coupled between the thermal control unit and the fluid-circulating blanket,
wherein the thermal control unit comprises:
a fluid reservoir;
a non-magnetic pump configured to pump the fluid through the fluid tubing to the plurality of fluid flow channels of the fluid-circulating blanket;
a first non-magnetic ultrasonic motor coupled to the non-magnetic pump, the first ultrasonic motor configured to control operation of the non-magnetic pump;
a heating and cooling assembly coupled between the fluid reservoir and the non-magnetic pump, the heating and cooling assembly configured to heat or cool the fluid to a specified temperature;
a heat sink coupled to the heating and cooling assembly;
a fan configured to provide air flow to the heat sink; and
a second non-magnetic ultrasonic motor coupled to the fan, the second non-magnetic ultrasonic motor configured to control operation of the fan.

11. The MRI-safe patient thermal management system of claim 10 further comprising an interface coupled to the thermal control unit comprising:
a display configured to display a temperature reading of the patient;
one or more actuable inputs configured to enable an operator to change operating parameters of the system.

12. The MRI-safe patient thermal management system of claim 10, wherein the heating and cooling assembly comprises a thermoelectric cooler.

13. The MM-safe patient thermal management system of claim 10, further comprising a power supply configured to provide power to the non-magnetic ultrasonic motor.

14. The MRI-safe patient thermal management system of claim 13, wherein the power supply is configured to supply a substantially sinusoidal alternating current with minimal harmonic frequencies in the range of about 6 MHz to about 130 MHz.

15. An MRI-safe patient thermal management system comprising:
a fluid-circulating blanket having a plurality of fluid flow channels;
a thermal control unit configured to control a temperature of fluid to be delivered to the plurality of fluid flow channels; and
fluid tubing coupled between the thermal control unit and the fluid-circulating blanket,
wherein the thermal control unit comprises:
a non-magnetic pump configured to pump the fluid through the fluid tubing to the plurality of fluid flow channels of the fluid-circulating blanket; and
a heating and cooling assembly coupled between the fluid-circulating blanket and the non-magnetic pump, the heating and cooling assembly configured to heat or cool the fluid to a specified temperature.

16. The MRI-safe patient thermal management system of claim 15 further comprising a non-magnetic ultrasonic motor coupled to the non-magnetic pump, the ultrasonic motor configured to control operation of the non-magnetic pump.

17. The MRI-safe patient thermal management system of claim 15 further comprising an interface coupled to the thermal control unit comprising:
a display configured to display a temperature reading of the patient;
one or more actuable inputs configured to enable an operator to change operating parameters of the system.

18. The MM-safe patient thermal management system of claim 15, wherein the heating and cooling assembly comprises a thermoelectric cooler.

19. The MRI-safe patient thermal management system of claim 18 further comprising:
a heat sink coupled to the heating and cooling assembly;
a fan configured to provide air flow to the heat sink; and
a non-magnetic ultrasonic motor coupled to the fan, the non-magnetic ultrasonic motor configured to control operation of the fan.

20. The MRI-safe patient thermal management system of claim 15, wherein the non-magnetic pump comprises a piezoelectric diaphragm pump.
